(19) United States
(12) Patent Application Publication
(10) Pub. No.: US 2025/0359819 A1
(43) Pub. Date: Nov. 27, 2025

(54) WEARABLE MONITORING SYSTEM

(71) Applicant: TRAQBEAT TECHNOLOGIES IKE, AGIOS MYRONAS (IRAKLEIOU) (GR)

(72) Inventors: EMMANOUIL SPANAKIS, AGIOS MYRONAS (IRAKLEIOU) (GR); EVANGELOS SAKKALIS, ATHENS (GR); MATTHAIOS PEDIADITIS, HERAKLION CRETE (GR)

(73) Assignee: TRAQBEAT TECHNOLOGIES IKE, AGIOS MYRONAS (IRAKLEIOU (GR)

(21) Appl. No.: 18/875,127

(22) PCT Filed: Jun. 15, 2023
(86) PCT No.: PCT/EP2023/066112

§ 371 Date: Dec. 14, 2024

(30) Foreign Application Priority Data
Jun. 15, 2022 (GR) 20220100495

(51) Int. Cl. A61B 5/00; A61B 5/1455; A61B 5/256; A61B 5/257; A61B 5/282; A61B 5/318

(52) U.S. Cl. CPC A61B 5/6824; A61B 5/14551; A61B 5/256; A61B 5/257; A61B 5/282; A61B 5/318; A61B 5/6823; A61B 5/6833; A61B 5/6838; A61B 2560/0443; A61B 2560/0468; A61B 2562/0233; A61B 2562/227

(57) ABSTRACT

A device (1) for measuring and/or monitoring physiological signals of a user configured to be attached to a first (12) and a second (13) wearable holder, wherein each of the first (12) and second (13) wearable holders is configured to be attached to a different part of a user's body, and the first wearable holder (12) comprises a first holding frame (14, 41) configured to removably be attached to the device (1) and to wrist fastening means.

### TECHNICAL FIELD

The present disclosure concerns a wearable device for measuring and/or monitoring physiological signals and a system comprising wearable holders configured to be attached to different parts of a user's body.

### BACKGROUND

Wearable devices include electronic devices designed to collect data about users' personal health. Driven by the increasing demand of consumers to monitor their own health and keep track of their own vital signs, the use of wearable technology has more than tripled in the last four years.

However, wearable devices often do not meet the required standards in healthcare, most of them being developed as fitness tracking devices. In other words, wearable devices usually do not achieve the required accuracy as to be implemented for reliably monitoring vital and/or relevant physiological signals.

The monitoring of physiological signals in healthcare is a critical technological aspect of clinical care. These signals normally represent parameters related to the physiological function of the circulatory, neurological, and respiratory systems. Therefore, adequate data collection may require signal's measurement on different body areas. However, current wearable devices comprise a limited functionality; there are not examples of wearable devices or systems which can be readily and technically adapted to monitoring on different parts of the body. In light of the above, wearable devices or systems capable of providing accurate and reliable information about the patient's health status while, according to the needs, having enough versatility for them to be applied to data collection on different parts of the body are required. The present invention addresses these needs and problems.

### SUMMARY OF THE INVENTION

In accordance with various embodiments of the invention, a wearable device for measuring and/or monitoring physiological signals and a system comprising wearable holders configured to be attached to different parts of a user's body are provided.

A first aspect of the invention concerns a device for measuring and/or monitoring physiological signals of a user configured to be attached to a first and a second wearable holder, wherein each of the first and second wearable holders is configured to be attached to a different part of a user's body. In other words, the device is configured to be attached to each of the first and second wearable holders. Therefore, the device allows to measure physiological signals, e.g., vital signals, on different parts of the body. For instance, the device may be configured to be attached to a first wearable holder, the first wearable holder being in turn configured to be attached to a wrist of a user; and also be adapted to be connected to a second wearable holder configured to be attached to the user's chest.

In a preferred embodiment of the first aspect of the invention, the first wearable holder comprises a first holding frame configured to removably be attached to the device and to wrist fastening means. More preferably, the first wearable holder is configured to be attached to a wrist of the user's body; the first holding frame comprises a side which remains closer to the user's skin when the first wearable holder is attached to the wrist; and the first holding frame is further configured for introducing to the same the device through said side.

In a preferred embodiment, the device is interchangeable between the first and second wearable holders. Hence, the device preferably can be removed, i.e de-attached, from the first wearable holder and be removably placed, i.e. be attached to, the second wearable holder, and vice versa.

In preferred embodiments, the device is configured to perform any of a SpO2 measurement, a Photoplethysmography (PPG), a Galvanic Skin Response (GSR), an electrodermal activity or a combination thereof.

In a preferred embodiment, the device is configured to be removably connected to external electrodes, preferably to electrocardiogram (EGC) electrodes. More preferably, the device comprises pins for connecting said optional external electrodes to the device when the latter is attached to the second wearable holder.

In preferred embodiments, the device comprises pairs of radiation sources which are light emitting diodes (LEDs), wherein said pairs comprises any, or all of, a pair of green LEDs, a pair of yellow-green LEDs, a pair of red LEDs. More preferably said red LEDs are dual red/infrared LEDs configured to emit red and infrared electromagnetic radiation. Very preferably, said radiation sources are arranged around an optional photodiode area of the device.

In an embodiment, the device is adapted to perform continuous and synchronous monitoring of patients' vital or physiological signals. The device may be able to provide information and/or data about or related with the cuffless blood pressure, heart rate, heart rate variability, respiratory rate, oxygen saturation (SpO2), skin temperature, one-lead electrocardiograph and/or activity tracking, among others.

The device in one embodiment comprises charging means such as one or more batteries. Therefore, the device may be a battery powered device. The device may allow for several days monitoring of the physiological signals on a single charge, preferably between 3-7 days monitoring on a single charge.

In a preferred embodiment, the device comprises: a casing comprising a first portion which comprises one or more holes extending between two opposite to each other surfaces, and a first electrode; and a second portion comprising an internal and an external side which are opposite to each other, one or more holes that extend between the external and the internal sides, and a second electrode attached to the external side; and wherein the first and second portions of the casing define a hollow cavity; and a printed circuit board, preferably an optoelectronic circuit board, to be fitted in the hollow cavity, comprising one or more radiation sources and/or one or more sensors configured to detect one or more signals. The above configuration makes the device adequate for accurately measuring or monitoring physiological signals while reducing the space occupied by the device.

In a preferred embodiment the device comprises a casing which can be removably and securely attached to the first wearable holder and to the second wearable holder such that the first and second holders are interchangeable for use with the device. Therefore, advantageously the casing of the device of said preferred embodiment may allow for easily detaching the casing, and hence the device, from the first wearable holder with which the device may be initially used, then moving and securely attaching, i.e. fixing in a stable manner, the device to the second wearable holder for using again the device in combination with the second wearable holder. Hence, in the latter preferred embodiment advantageously the first and second wearable holder exhibit an excellent and optimized interchangeability in the sense that one of the two holders can easily replace the other for wearing the device on the human body. This interchangeability may in turn allow optimizing specific applications or particular embodiments of the invention. For example, an embodiment wherein the device allows taking ECG measurements, the first wearable holder allows wearing the device on the wrist of the user, and the second wearable holder comprises a patch for wearing the device on the chest of the user, interchanging easily the first with the second wearable holder for changing from using the device on the wrist to using the device on the chest, may allow for more accurate (motion artifact free) taking of the ECG measurement(s), especially when said measurements are to be taken when the user is sleeping. However, the first wearable holder for the wrist provides a more convenient way for the user/patient even if more error prone due to motion artifacts, but still there is the need to use the hand opposite the holder and hold your finger on the sensor designated metallic contact; something that is not needed in the case of the second holder (patch version). Hence, the patch version is preferable for short-term highly accurate measurements, whereas the wrist version for a more convenient long-term measurements but with variable accuracy.

A second aspect of the invention concerns a wearable system comprising: a first and a second wearable holder, wherein each of the first and second wearable holders is configured to be attached to a different part of a user's body; and a device according to the first aspect of the invention, configured to be attached to the first and second wearable holders. The device is thus interchangeable between the first and second wearable holders, resulting in that the wearable system allows for monitoring on different parts or areas of the body.

In a preferred embodiment, the first wearable holder is configured to be attached to a wrist of the user's body, and the second wearable holder is configured to be attached to a chest of the user's body.

In an embodiment, the first wearable holder comprises a first holding frame configured to removably attach (to the same) the device and wrist fastening means. The first holding frame, i.e., a first frame which holds the device, may be therefore configured to be attached to a user's wrist. In this embodiment, the device of the system, when attached to the first wearable device, i.e., to the first holding frame, allows for measuring or monitoring physiological signals specifically from an area of the user's wrist.

In preferred embodiment of a system according to the second aspect of the invention, the system further comprises the aforementioned wrist fastening means.

Advantageously, the second wearable holder may comprise a patch. The patch may be adapted to be attached to a user's chest. When attached to a user's chest the device may measure or monitor physiological signals specifically from an area of the user's chest.

In a preferred embodiment, the second wearable holder comprises a patch, and said patch comprises one or more electrocardiogram (EEG) external electrodes.

In other embodiments, the patch may comprise apertures extending between two opposite to each other surfaces of the patch. The apertures may leave free areas on the user's skin. In further embodiments, the patch may comprise one or more channels or grooves. The channels or grooves may be configured to introduce cables through them for connecting one or more off-the-shelf standardized single use electrocardiogram (ECG) patches.

In another embodiment, the second wearable holder further comprises an adhesive layer attached to the patch and a second holding frame configured to removably attach the device. The adhesive layer, e.g., a double-sided adhesive tape, may allow the patch to be removably attached to a user's chest. The second holding frame i.e., a second frame which holds the device, may be connected and/or integral to the patch.

In a further embodiment, the first and second wearable holders comprise one or more snap-in pockets, and the device comprises one or more protrusions, or vice versa; the protrusions and snap-in pockets being configured to fit each other. The protrusions and snap-in pockets may allow to removably attach the device to the first and second wearable holders.

In a preferred embodiment, the first wearable holder comprises a first holding frame, a clamp which is curved, forms a single piece and protrudes from the first holding frame, and the first wearable holder also comprises a curved rigid element that is hingedly connected to the first holding frame; the curved rigid element can pivot about the first holding frame so that an end of the curved rigid element can go around and be attached to a surface of the clamp; and the curved rigid element, the first holding frame and the clamp form a bracelet when the end of the curved rigid element is attached to the surface of the clamp. It is contemplated that advantageously one, e.g. a first responder, may easily place the first wearable holder of the latter preferred embodiment on a wrist of another person (e.g. on the wrist of a patient or victim of an accident), and in fact may do so single handedly with one hand, which can be particularly desirable in time critical situations, e.g. in mass-casualty events.

In a preferred embodiment which is according to the previous one, the wearable system further comprises hook-and-loop means via (i.e. be means of) which the end of the curved rigid element can be attached to and can be removably fastened to the surface of the clamp.

In a preferred embodiment which is according to the previous one, the clamp comprises a bended portion at an end of the clamp, said end of the clamp being on/at an opposite side of the clamp compared to the first holding frame, wherein said bended portion is curved oppositely (i.e. towards an opposite direction) compared to the rest of the clamp.

Various modifications and additions can be made to the embodiments discussed without departing from the scope of the invention. For example, while the embodiments described above refer to particular features, the scope of this invention also includes embodiments having different combination of features and embodiments that do not include all of the above-described features.

### BRIEF DESCRIPTION OF THE DRAWINGS

A further understanding of the nature and advantages of particular embodiments may be realized by reference to the remaining portions of the specification and the drawings, in which like reference numerals are used to refer to similar components.

FIG. 1 is a rear perspective view of an embodiment of a device according to the invention;

FIG. 2 is an upper perspective view of an embodiment of a device according to the invention;

FIG. 3 is a rear exploded view of an embodiment of a device according to the invention;

FIG. 4 is an upper exploded view of an embodiment of a device according to the invention;

FIG. 5 is a rear perspective view of an embodiment of a second wearable holder according to the invention;

FIG. 6 is an upper perspective view of an embodiment of a second wearable holder according to the invention;

FIG. 7 is a rear perspective view of an embodiment of a first wearable holder according to the invention;

FIG. 8 is an upper perspective view of an embodiment of a first wearable holder according to the invention;

FIG. 9 is a rear view of an embodiment of a printed circuit board according to the invention;

FIG. 10 is a front view of an embodiment of second wearable holder according to the invention attached to a users' chest.

FIG. 11 is a rear perspective view of an embodiment of a second wearable holder according to the invention;

FIG. 12 is an upper perspective view of an embodiment of a second wearable holder according to the invention; and

FIG. 13 is an upper view of an embodiment of a second wearable holder according to the invention.

FIG. 14 is a view of an embodiment of a first wearable holding according to the invention.

FIG. 15 is a side view of the embodiment of FIG. 14.

### DETAILED DESCRIPTION OF EMBODIMENTS

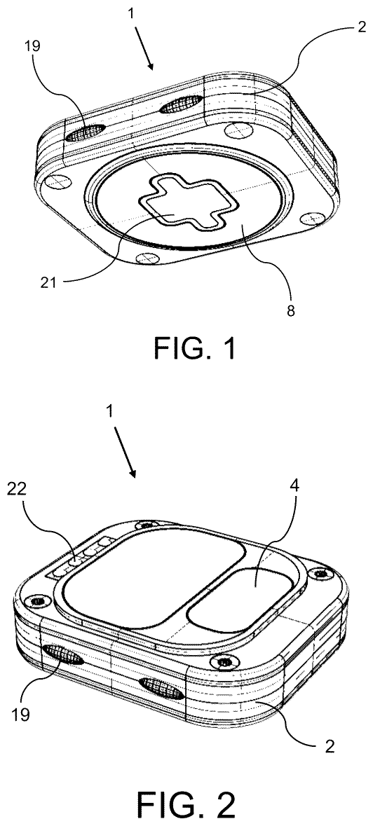

Reference is made to FIG. 1-2, which show an embodiment according to the first aspect of the invention. The embodiment corresponds to a device 1 for measuring and/or monitoring physiological signals of a user. The device is configured to be attached to a first 12 and a second 13 wearable holder, wherein each of the first 12 and second 13 wearable holders is configured to be attached to a different part of a user's body. The device comprises a casing 2.

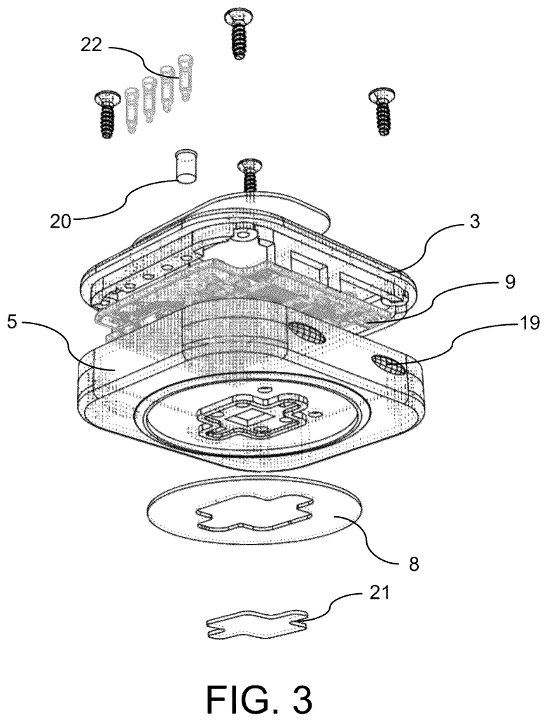

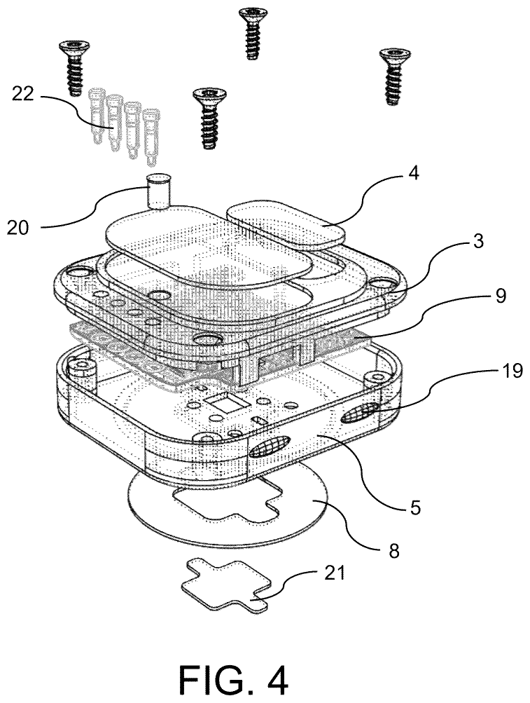

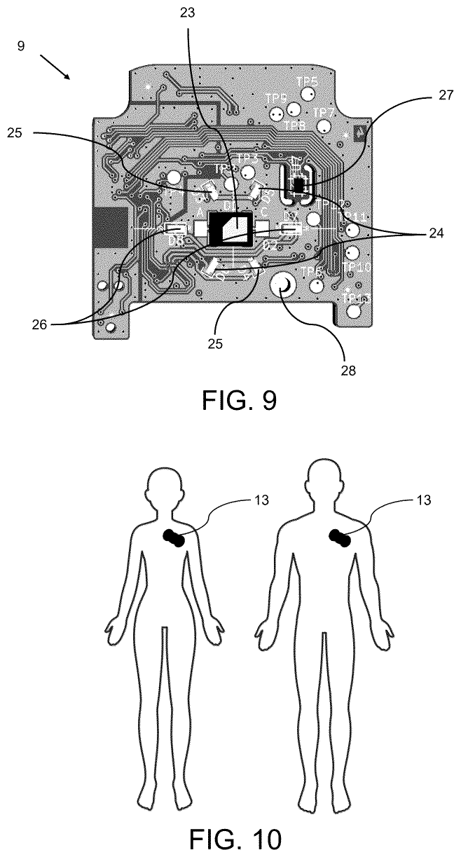

In FIG. 3-4 there are provided exploded perspective views of an embodiment according to the first aspect of the invention. As it is shown, the device may comprise a casing comprising a first portion 3 which comprises one or more holes extending between two opposite to each other surfaces, and a first electrode 4; and a second portion 5 comprising an internal 6 and an external side 7 which are opposite to each other, one or more holes that extend between the external and the internal sides, and a second electrode 8 attached to the external side; and wherein the first 3 and second 5 portions of the casing define a hollow cavity; and a printed circuit board 9, preferably an optoelectronic circuit board, to be fitted in the hollow cavity. It is contemplated that the printed circuit board (PCB) comprises one or more radiation sources and/or one or more sensors configured to detect one or more signals, as shown in FIG. 9. The first and second portion may be attached to each other or assembled by any means known in the art such as gluing, screwing, as shown in embodiments of FIG. 1-4, snap-in tabs or flanges or others. The first portion of the casing may correspond to the upper portion of the casing, i.e., the portion of the casing that is further away from the user's skin while the device is being used. The holes extending between two opposite to each other surfaces of the first portion of the casing may be used for fitting in or introducing elements which provide information to the user such as indicators 20 (e.g., LED indicators), labels, displays; or other technical elements such as the first electrode 4. The one or more holes that extend between the external and the internal sides of second portion of the casing may be used for fitting in or introducing the one or more radiation sources and/or one or more sensors to be connected to the PCB.

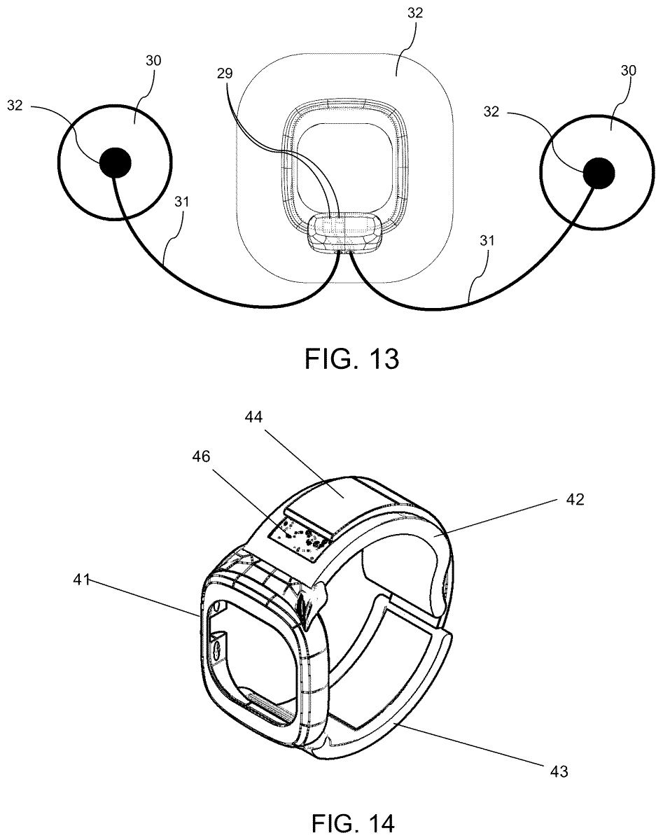

As shown in FIG. 3-4, the second electrode of the second portion of the casing may comprise a hole that allow to the one or more radiation sources and sensors to actuate for the measuring and/or monitoring of the physiological signals. It is to be understood that the second electrode may also comprise more than one hole for this purpose. The first and second electrodes may have any shape or size as long as they provide an adequate contact with the surface of the user's skin. Further, the hole of the second electrode is used for fitting in a transparent element 21 or material, such as for instance a lamellar element made of polycarbonate. Said transparent element 21 allows to perform through it optical measurements by use of radiation sources and optical sensors, while it provides a physical separation between the external side of the second portion of the casing and the user's skin. The radiation sources may be any of those known in the art, such as LEDs, and the one or more sensors may comprise temperature sensors, optical sensors, electrical sensors, pressure sensors, proximity sensors, infrared sensors, humidity sensors, tilt sensors, vibration sensors or others. As shown in FIG. 2-4 the device may comprise pins 22, which can also be called contact points, for charging and connecting external electrodes 30, such as EGC electrodes. The external electrodes 30 can also be used for measuring Galvanic Skin Response (GSR), also known as electrodermal activity (EDA). The four pins 22 of the device may serve the following purposes: two of them are used for charging the device, e.g., the device may be inserted into a charger, which has two contacts for contacting the two charging points; and the other two pins or contact points may be used for connecting to ECG electrodes when the device is attached to a second wearable holder that is a patch. To this aim, the patch frame, i.e., the second holding frame, may comprise two spring contacts 29 positioned over the respective points on the device, as shown in FIG. 13. The spring contacts 29 may be held in place through a PCB. When the main unit is inserted into the patch frame, these two spring contacts 29 touch, or contact, and connect two external electrodes, which preferably are single use electrodes. Each external electrode 30 may be connected to a spring contact using a wire 31 and/or a flexible PCB. Further, each external electrode 30 may be connected through a 3.5- or 4-mm ECG snap button connector 32.

In some embodiments where the device is attached to a second wearable holder, and the second wearable holder is a patch, the switching between the external electrodes may be realized using an electronic double pole double throw switch, wherein each pole can switch independently. Based on this, different configurations may be adopted: a first configuration wherein a first external electrode and a second external electrode are connected to an EGC analog digital frontend, which is the interface between physical signals and a digital processor; a second configuration wherein the first external electrode and the second electrode, the latter being attached to the external side of the second portion of the casing, are connected to the EGC analog digital frontend; and a third configuration wherein the second external electrode and the second electrode are connected to the EGC analog digital frontend. The first configuration provides the best signal-to-noise ratio (SNR), while the second and third configurations may be used in case the electrode contact is deteriorated. In some embodiments, the configuration may be set by a controlling application (e.g., on mobile phone) according to the use-case, which may be set manually by the user. In other embodiments, the configuration may also be set automatically using a built-in ECG lead-off detection mechanism. In this case, the switch may cycle through each configuration, while the lead-off detection mechanism tests each electrode to check if it has contact to the skin. This testing cycle may also be enabled if, while using the first configuration a deterioration in electrode contact occurs (detected trough the lead-off mechanism). Therefore, in this case, the second and third configurations may be used.

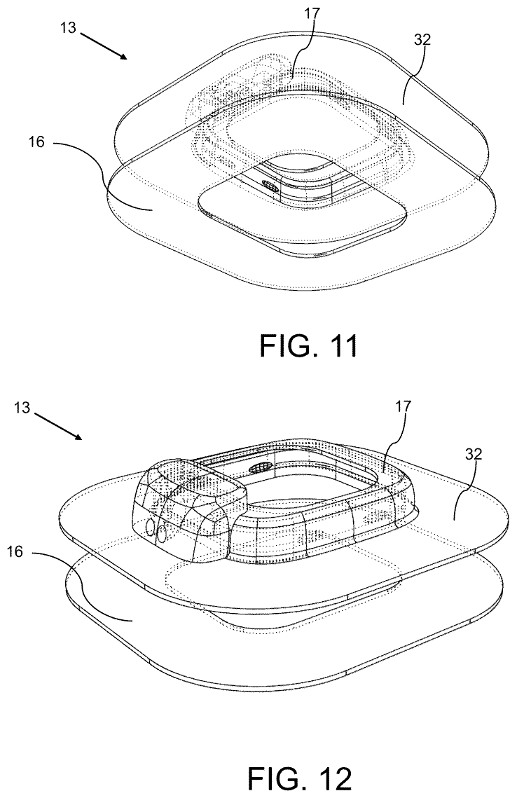

In the embodiment of FIG. 13, which comprises the second wearable holder of FIG. 11-12, there are shown EGC external electrodes 30 connected to the wires 31 using snap buttons 32. This embodiment allows for a flexible selection of the EGC electrodes, as explained above.

In other embodiments where the device is attached to the first wearable holder, the first and second electrodes may be connected to the ECG analog digital frontend.

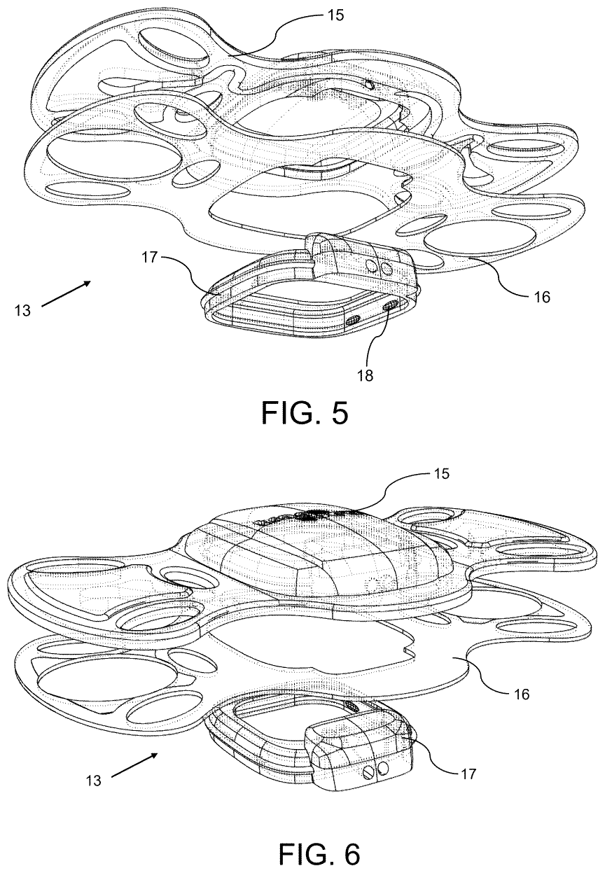

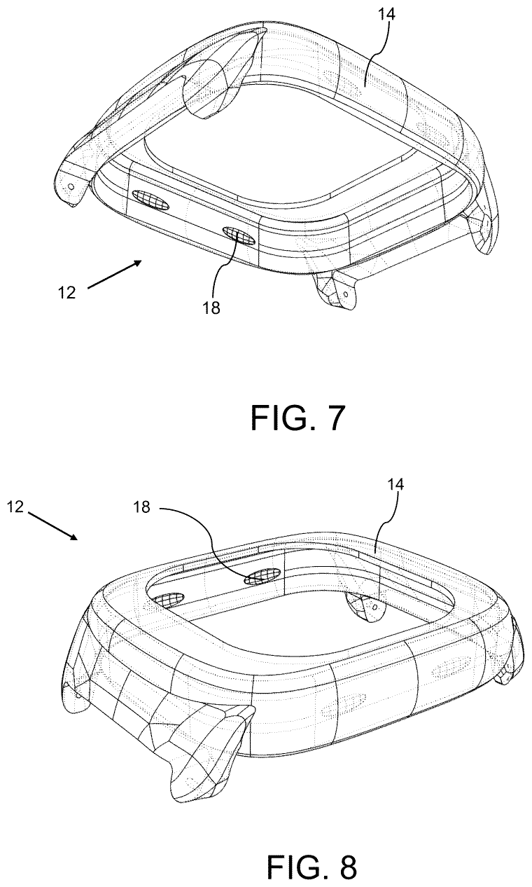

The system according to the second aspect of the invention is separately depicted in different figures. For instance, FIG. 5-6 show an embodiment of a first wearable holder, whilst FIGS. 7-8 and 11-13 show different embodiments of a second wearable holder, and embodiments of the device are shown in FIG. 1-4. However, it is to be understood that the system comprises a first 12 and a second 13 wearable holder, wherein each of the first 12 and second 13 wearable holders is configured to be attached to a different part of a user's body; and a device 1 according to the first aspect of the invention, configured to be attached to the first 12 and second 13 wearable holders.

FIGS. 5 and 6 show an embodiment of a second wearable holder 13 wherein the second wearable holder 13 is a patch 15. The patch comprises apertures extending between two opposite to each other surfaces of the patch. The apertures may leave free areas on the user's skin and thus, reduce the sweating when the patch is attached or sticked to the user's skin. As shown in FIG. 13, the patch may also comprise one or more electrocardiogram (EGC) external electrodes, i.e., conductive pads that are attached to the skin and enable recording of electrical currents. Further, the patch may be made of an elastic material which improves its adaptability to the user's skin, being stretchable and bendable. As it is shown, the second holding frame is configured to introduce the device through a bottom side of the second holding frame, i.e., the side of the frame which remains closer to the user's skin when the second wearable holder is used. Therefore, the device will not fall out of the second holding frame. The device may also be protected by an elastic part that covers it. This may provide extra protection making the pins to electrode contacts watertight. FIG. 10 shows an embodiment of a device attached to a second wearable holder, which is attached or sticked to a users' chest. In this particular embodiment, the second wearable holder is designed to be positioned on the left side of the chest, above the left breast at an angle of 25-45 degrees with respect to the horizontal axis.

The embodiment of the second wearable holder in FIGS. 5 and 6 comprises one or more channels or grooves which are configured to introduce cables through them for connecting the ECG electrodes. In this embodiment, the second wearable holder 13 further comprises an adhesive layer 16, which may be configured as a double-sided adhesive tape, attached to the patch and a second holding frame 17 configured to removably attach the device 1. The adhesive layer 16 may be made of an acrylate specially designed for medical/surgical use for sticking the patch to the user's skin. The tape may be cut by a Computer Numerical Control (CNC) machine to provide the tape with a particularly advantageous shape for allowing the introduction of electrodes therein and/or reduce the sweating when the tape is sticked to the user's skin. As shown in the figures, the adhesive layer may also comprise holes for fitting EGC electrodes in the second wearable holder. The first and second holding frames shown in FIG. 5-8 are made of a rigid material, preferably a rigid plastic material.

Regarding FIGS. 7 and 8, there is shown an embodiment of a first wearable holder wherein the first wearable holder 12 comprises a first holding frame 14 configured to removably attach the device 1 and wrist fastening means (the latter not shown). As it is shown, the first holding frame is configured to introduce the device through a bottom side of the first holding frame, i.e., the side of the frame which remains closer to the user's skin when the first wearable holder is used. Therefore, the device will not fall out of the first holding frame. The first holding frame is connected or attached to the wrist fastening means (the latter not shown). In alternative embodiments, it is contemplated that, when the user is wearing the first wearable holder, it is not necessary to remove or detach the first wearable holder from the user's wrist for attaching or detaching the device.

As shown in FIG. 1-8, the first 12 and second 13 wearable holders comprise one or more snap-in pockets 18, and the device comprises one or more protrusions 19. However, it is to be understood that a configuration where the wearable holders comprise one or more protrusions and the device comprise one or more snap-in pockets is also possible. The protrusions 19 and snap-in pockets 18 are configured to fit each other and thus, they contribute to facilitate the removable attachment of the device to the wearable holders.

Reference is now made to FIG. 9, wherein an embodiment of a PCB 9 is shown. The embodiment of FIG. 9 comprises one or more radiation sources and/or one or more sensors configured to detect one or more signals, wherein the radiation sources are LED light sources with 4 different wavelengths (green LED 24, yellow-green LED 25, red/IR LED 26) arranged around a photodiode 23 area. Although the PCB may comprise one or more LEDs, in this embodiment there are shown two pairs of LEDs for each of the wavelengths: 530 nm (green), 570 nm (yellow-green), 655 nm (red), and 940 nm (IR). In this particular embodiment, the red/IR LEDs are dual LEDs arranged such that the IR emission sources are at a further distance from the photodiode than the red, green and yellow-green LEDs. This provides an even tissue penetration, compensating the weaker IR light absorption of the tissue. The PCB also comprises a temperature sensor 27 and a spring connector 28 for placing an ECG electrode through the hole.

Hemodynamic effects can be monitored non-invasively using the Photoplethysmography (PPG), which is a simple and low-cost optical technique that can be used to detect blood volume changes in the microvascular bed of tissue. Conventional PPG measurement devices measure transmissive PPG at the finger using light sources at 655 nm (red), and 940 nm (IR). At these locations, said two wavelengths are used together for SpO2 measurements. At the wrist and at the chest, reflective PPG is used, where these two wavelengths may offer a high dynamic component but are very sensitive against motion due to the higher tissue penetration. Green is commonly used for wrist PPG and offers the best overall SNR in scenarios with and without motion. Yellow-green has similar properties to green but due to its different wavelength it allows for the measurement of SpO2, which needs two wavelengths. Therefore, the present invention may also comprise green and yellow-green colors for reflective PPG.

In some embodiments, the device comprises LEDs for each of the wavelengths: 530 nm (green), 570 nm (yellow-green), 655 nm (red), and 940 nm (IR). In this way, it is achieved different tissue penetration depths. The first two extent from the superficial capillary bed (Green) to the arterioles in the dermis (Yellow-Green), while Red and IR can penetrate much deeper reaching the arteries in the subcutaneous tissue. It should be noted that conventional approaches using a single red or IR sensor often lead to inaccurate results especially in estimating pulse transit time (PTT) since the PPG signal captured is a superposition of functions with different waveforms and phase shifts. Having all four wavelengths available in the configuration shown in FIG. 9 further enhances the performance of the device. Further, in some embodiments, it is possible to dynamically choose the best wavelength/penetration depth given the application (patch/wrist) and measurement conditions (skin type/placement) using an algorithm based on signal quality estimation metrics such as kurtosis, SNR and zero-crossing rate.

The embodiments wherein the device comprises LEDs of four wavelengths allow to estimate the constant amounts of light absorbed from body tissues, such as bones, muscles, venous blood, and melanin using the Green/Yellow-Green LEDs and estimate the most critical information reflecting the pulsatile changes in arterial blood volume using the Red/IR light sources. The former produces the constant direct component (DC), whereas the later produces the information-rich alternating component (AC) of PPG.

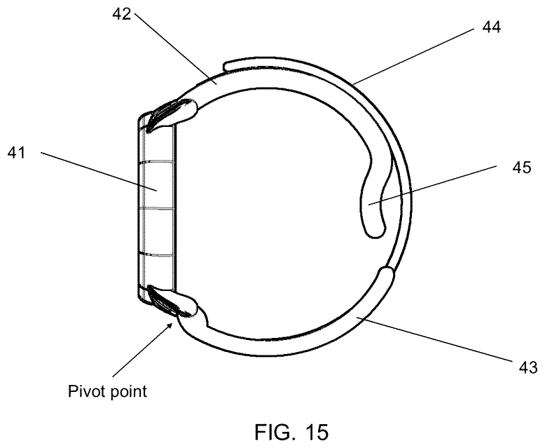

As to the FIG. 11-12, there is shown an embodiment of a second wearable holder 13 which comprises a double-sided adhesive layer 16 attached to the patch and a second holding frame 17 configured to removably attach the device 1. In this embodiment, the device comprises a flange which extending from the second holding frame for facilitating the adhesion to the user's skin and the placement of the adhesive layer. Further, in other embodiments where the double-sided adhesive layer is not present, it is contemplated that the patch may be attached to the user's skin by using common single-sided medical tape covering the flange of the second wearable holder. FIG. 14 and FIG. 15 show a first wearable holder of a preferred embodiment of a system according to the second aspect of the invention. The wearable holder shown in FIG. 14 and FIG. 15 is configured to be attached to a wrist of a user, and comprises a first holding frame 41 which comprises an arm clamp/hook 42 which is rigidly connected to the frame and forms with the latter a single piece. The (first) holding frame of 41 may also be called sensor frame because it is configured for receiving and be removably be attached to a device which is according to the first aspect of the invention. In the embodiment of FIG. 14 said clamp 42 (hook) is curved and circularly protruding from the frame and is flexible. Alternatively, said clamp instead of forming a single piece with the holding frame 41, may be removably connected to the latter, e.g. via a hinge or other connection means. In the embodiment of FIG. 14, said clamp 42 comprises a first strip comprising an exposed rough surface 46. The first strip is part of (i.e. is comprised by) a hook-and-loop fastening means of the embodiment of FIG. 14. Said rough surface 46 is opposite to an inner surface/side of the clamp 42, said inner surface/side being the one that is closer to the user's skin when the first wearable holder of FIG. 14 is worn on the user's wrist. An end of the clamp 2, said end being opposite to the clamp's side/part connected to the holding frame 41, comprises a bended portion 45. As shown in FIG. 15, said bended portion 45 is curved oppositely compared to the rest of the clamp 42. Also, said bended portion facilitates hooking the first wearable holder on the wrist, and also promotes the stability of the wearable holder on the wrist when said holder is placed on the wrist. The first wearable holder of FIG. 14 further comprises a curved rigid element 43 which is connected to the sensor frame 41 through a hinge mechanism, enabling it to pivot freely. A pivot point about which the rigid element 43 of FIG. 14 can pivot with respect to the first holding frame 41, is indicated in FIG. 15. At an end 44 of the curved rigid element 43 of FIG. 14, there is attached a second strip such that a smooth surface of the second strip is exposed. The second strip is also part of the hook-and-loop fastening means of the embodiment of FIG. 14, and can be removably attached to the rough surface 46 of the first strip. In the embodiment of FIG. 14, the curved rigid element 43 can pivot about the pivot point so that the end 44 comprising the second strip can go around the clamp/hook 42 and be attached to the rough surface 46 of the first strip, for securely and removably fastening on the wrist the bracelet that is formed by the holding frame 41, the curved rigid element 43 and the clamp 42. It can be understood that in the embodiment of FIG. 14 the rigid pivoting element 43 is a type of wrist fastening means removably connected to the first holding frame 41. Also, it can be understood than in the embodiment of FIG. 14 the rigid pivoting element 43 advantageously serves as a means to support the sensor frame 41 for keeping it at a correct location at the wrist when the bracelet is worn. Also the rigid pivoting element 43 advantageously serves as a “handle” which the person can use to hold and use as guide for correctly and quickly placing the sensor frame 41 at the wrist.

1. A device (1) for measuring and/or monitoring physiological signals of a user configured to be attached to a first (12) and a second (13) wearable holder, wherein each of the first (12) and second (13) wearable holders is configured to be attached to a different part of a user's body, and the first wearable holder (12) comprises a first holding frame (14, 41) configured to removably be attached to the device (1) and to wrist fastening means.

2. A device according to claim 1, wherein the device is interchangeable between the first and second wearable holders (12, 13).

3. A device according to claim 1, wherein the device is configured for performing any of a SpO2 measurement, a Photoplethysmography “PPG”, a Galvanic Skin Response “GSR”, an electrodermal activity or a combination thereof.

4. A device according to claim 1, wherein the device is configured to be removably connected to external electrodes, preferably to electrocardiogram “EGC” electrodes.

5. A device according to claim 4, wherein the device comprises pins (22) for connecting the external electrodes (30) to the device when the latter is attached to the second wearable holder (13).

6. The device according to claim 1, comprising:
a casing (2) comprising a first portion (3) which comprises one or more holes extending between two opposite to each other surfaces, and a first electrode (4); and a second portion (5) comprising an internal (6) and an external side (7) which are opposite to each other, one or more holes that extend between the external and the internal sides, and a second electrode (8) attached to the external side; and wherein the first (3) and second (5) portions of the casing define a hollow cavity; and
a printed circuit board (9), preferably an optoelectronic circuit board, to be fitted in the hollow cavity, comprising one or more radiation sources and/or one or more sensors configured to detect one or more signals.

7. A device according to claim 1, comprising pairs of radiation sources which are light emitting diodes “LEDs”, wherein said pairs comprises any, and preferably all of, a pair of green LEDs (24), a pair of yellow-green LEDs (25), a pair of red LEDs (26), more preferably the red LEDs being dual red/infrared LEDs configured to emit red and infrared electromagnetic radiation.

8. A device according to claim 7, wherein the radiation sources are arranged around a photodiode area (23) of the device.

9. A device according to claim 1, comprising a casing (2) which can be removably and securely attached to the first wearable holder (12) and to the second (13) wearable holder such that the first (12) and second (13) holders are interchangeable for use with the device.

10. A wearable system comprising:
a first (12) and a second (13) wearable holder, wherein each of the first (12) and second (13) wearable holders is configured to be attached to a different part of a user's body, and the first wearable holder (12) comprises a first holding frame (14, 41) configured to be removably attached to the device (1) and to wrist fastening means;
a device (1) according to claim 1, configured to be attached to the first (12) and second (13) wearable holders.

11. The wearable system according to claim 10, wherein the first wearable holder (12) is configured to be attached to a wrist of the user's body; the first holding frame (14, 41) comprises a side which remains closer to the user's skin when the first wearable holder is attached to the wrist; and the first holding frame (14, 41) is further configured for introducing to the same the device through said side.

12. The wearable system according to claim 11, wherein the first wearable holder is configured to be attached to a wrist of the user's body, and the second wearable holder is configured to be attached to a chest of the user's body.

13. The wearable system according to claim 10, wherein the second wearable holder (13) comprises a patch (15).

14. The wearable system according to claim 13, wherein the patch (15) comprises one or more electrocardiogram, EEG, external electrodes.

15. The wearable system according to claim 10, wherein the second wearable holder (13) further comprises an adhesive layer (16) attached to the patch and a second holding frame (17) configured to removably attach the device (1).

16. The wearable system according to claim 10, wherein the first (12) and second (13) wearable holders comprise one or more snap-in pockets (18), and the device comprises one or more protrusions (19), or vice versa; the protrusions (19) and snap-in pockets (18) being configured to fit each other.

17. The wearable system according to claim 10, further comprising the wrist fastening means.

18. The wearable system according to claim 10, wherein the first wearable holder (12) further comprises a clamp (42) which is curved, forms a single piece and protrudes from the first holding frame (41), and also comprises a curved rigid element (43) that is hingedly connected to the first holding frame (41); the curved rigid element (43) can pivot about the first holding frame (41) so that an end (44) of the curved rigid element (43) can go around and be attached to a surface of the clamp (42); and the curved rigid element, the first holding frame (41) and the clamp (42) form a bracelet when the end (44) of the curved rigid element is attached to the surface (46) of the clamp (42).

19. The wearable system according to claim 18, further comprising hook-and-loop means via which the end (44) of the curved rigid element is attachable to and can be removably fastened to the surface (46) of the clamp (42).

20. The wearable system according to claim 18, wherein the clamp (42) comprises a bended portion (45) at an end of the clamp, said end (45) of the clamp (42) being on an opposite side of the clamp (42) compared to the first holding frame (41), wherein said bended portion (45) is curved oppositely compared to the rest of the clamp (42).
